(19) United States
(12) Patent Application Publication
(10) Pub. No.: US 2024/0335214 A1
(43) Pub. Date: Oct. 10, 2024

(54) METHOD OF PROCUREMENT AND USE OF TISSUE FOR ALLOGRAFTS

(71) Applicant: Marcelo Andres FONSECA CANTEROS, Iquique (CL)

(72) Inventor: Marcelo Andres FONSECA CANTEROS, Iquique (CL)

(21) Appl. No.: 18/578,614

(22) PCT Filed: Jul. 14, 2021
(86) PCT No.: PCT/IB2021/056338

§ 371 Date: Jan. 11, 2024

(51) Int. Cl. A61B 17/322; A61L 27/36; A61L 27/60

(52) U.S. Cl. CPC A61B 17/322; A61L 27/3687; A61L 27/60; A61B 2017/3225

(57) ABSTRACT

This invention corresponds to a new alternative coverage for skin defects through the procurement of allografts of whole skin and the subsequent cryopreservation. The present invention seeks to provide a solution to the problem of temporary and definitive coverages of complex wounds. The proposed solution corresponds to procurement method, processing, and the subsequent use of the skin allografts for coverage of complex wounds.

### TECHNICAL FIELD

The present invention is part of the technical field of procurement and processing of tissues for transplants.

### BACKGROUND AND PREVIOUS ART

Extensive skin damage, produced mainly in burned patients, but also in other situations such as trauma and dermatological diseases which generate destruction and deepithelization of vast body segments, has forced the search for coverages and skin substitutes, having to emphasize that despite all current technological development, the autologous skin grafts continue to be the standard of treatment, mainly because there is no risk of rejection, thus achieving definitive coverage and solution. However, they present as a disadvantage the morbidity of the donor zone and the scarcity thereof, especially if there are large skin surfaces affected.

Cutaneous allografts (CA), which come from individuals of the same species, are an excellent substitute, as they fulfill the same functions of autologous skin, however, due to processes of immunity they are rejected, being only used as temporary coverage, and may eventually be a vector of infectocontagious diseases. There are currently a low number of donors and the logistics of the procurement, processing and storage of the skin for clinical use is complex; moreover, according to the latest general technical standard for the procurement, preservation and implantation of tissues of the Ministry of Health of Chile (MINSAL, February 2018) Only reserved for cadaveric donors, thus obtaining only limited partial thickness CA, with which there is an extensive clinical experience in the literature.

The multiple clinical benefits, but at the same time the shortage of CA, open the door for the search for potential skin donors, particularly in living donors who, because of their health condition, large amounts of their skin must be drying out for clinical improvement, as are those who undergone to body contour surgeries, for example abdominoplasties for esthetic and/or reconstructive reasons, so that they can obtain CA of whole skin (CAWS).

The clinical experience presented reaffirms the possibility that body contour surgeries will be transformed into CA sources, but in this particular case of total skin (CAWS), which, when preserved by cryopreservation (CAWSC), maintain their viability and many elements of the donor dermis, in particular, the fibroblasts which attach to the receptor, becoming in an intermediate coverage for some patients and definitive coverage for others.

In places where the rate of general and particular donation of skin is low and in others where its use is not yet legalized mainly for cultural and religious reasons, the use of CA from a living donor of patients undergone to body contouring procedures is opened as an option, in this particular case to an abdominoplasty. The number of effective skin donors in Chile in the 2017-2019 triennium was only four donors, obtaining 3,051 cm2, 1,069 cm2 and 6,839 cm2 of skin per year, respectively (all cadaveric donors). In our experience, approximately 302 cm2 clinically useful processed skin is obtained for each abdominoplasty, reflecting the potential of this procedure as an important source of CA and CAWS.

The present invention is a new alternative is a new alternative coverage for skin defects, temporary in some patients and definitive in others, which acts as a dermal regeneration matrix, which in some patients must be autografted to complete the process and in others can be managed with advanced healing to finish the healing process. It has three distinctive characteristics: a) they come from living donors, b) they include all the layers of the skin and c) they are preserved by cryopreservation, thus obtaining viable tissues.

The following describes state-of-the-art documents that are close to the invention.

US2020246508A1 describes scaffolding prepared from natural tissues which have been decellularized. It is indicated that this product is suitable for use as a tissue filler. The differences with the evaluated invention correspond to the fact that the tissue of the invention is not decellularized, no growth factors are added. In addition, its use appears not to be suitable for the treatment of major burns.

US2014316262A describes a method for identifying perforating veins to identify the ideal site for skin flap removal. The method consists in injecting a contrast medium and then identify the vein in the area through illumination. This document corresponds to the general state of art and does not affect the evaluated invention.

US2013108683A1 discloses methods and compositions for the treatment of wounds of varying severity and location. The composition corresponds to a biologically compatible matrix complemented by a growth factor. The claims indicate that this product serves to promote or improve transplants, such as skin flaps. The differences with the evaluated invention are clear, as this product would be used as a complement to the evaluated invention.

US2006073592A1 describes methods for tissue matrix storage. Specifically, dermis is mentioned, however, the method seeks to preserve decellularized tissues. Therefore, this document is considered to be the general state of art and it would not affect the invention evaluated.

WO0052149A1 In this case, protection is sought for a method of applying biological material to a patient. Initially it refers to cells, however, there is an alternative for using tissues, and skin flaps are mentioned in tissue examples.

The difference with the evaluated invention is that in this case the conditions for transplanting flaps are not specified.

### SUMMARY OF THE INVENTION

This invention corresponds to a new alternative coverage for skin defects through the procurement of allografts of whole skin and the subsequent cryopreservation.

### DETAILED DESCRIPTION OF THE INVENTION

The present invention seeks to provide a solution to the problem of temporary and definitive coverages of complex wounds.

The proposed solution corresponds to procurement method, processing, and the subsequent use of the skin allografts for coverage of complex wounds.

The method comprises 3 main stages, namely (i) obtaining whole skin tissue from a living donor, (ii) treatment and preservation of tissue, obtaining skin allograft, and finally (iii) grafting of the CAWS in the final patient.

In a more specific embodiment, stage i) of the method comprises the steps of a) surgical intervention, b) tissue procurement, c) packaging and identification, d) storage, and e) transport to the processing center.

On the other hand, the tissue treatment and conservation stage comprises the steps of f) processing, g) cryopreservation and quarantine, h) validation, records.

Finally, the stage that corresponds to the final process comprises the steps of i) transport and j) clinical use.

For more specific embodiments, step a) is performed in the ward, from where the tissue from the donor will be removed. In a specific step a marking is made of the area where the tissue will be dissected. Once it has been marked, the dissection of the adipose skin tissue is performed. Once said adipose skin tissue was obtained, the procurement itself is carried out.

In step b) the tissue obtained in step a) is moved to a suitable surface where whole skin procurement is performed, including the dermis, and releasing the fat from the deeper dermis using appropriate tools. As a quality control, in this step tissue samples are also taken for cultures of aerobic and anaerobic microorganisms, as well as mycotic cultures. Finally, the tissue is deposited in a sterile container with physiological serum. Optionally, the physiological serum may contain antibiotics. The container is hermetically sealed, ensuring that the tissue is completely immersed in the serum.

In stage (c) the containers obtained in stage (b) are stored in a sterile manner, labeling the container with the data of at least one code assigned to the tissue, date and time of procurement.

In d) the tissue is stored in a suitable container to maintain a temperature between 2 and 8° C. Subsequently, in e) the tissue is transported as quickly as possible to the processing center, within a period of less than 36 hours, maintaining the temperature in the range of 2 to 8° C.

Once the tissue arrives at the processing center, it undergoes to the stages: 1. Measurement of CAWS, segments cuts according to requisition, review of the shave, wash cycles to decrease microbial load, sample collection number 2 for and immersion in cryopreservation solution for a period of 30 minutes to 3 hours, 2. Preparation, trimming, measurement, packaging and labeling of each obtained film, together with sample collection 3 and 4 (culture) during packing stage and sample collection 5 (culture) for backing.

In step g), for cryopreservation and quarantine, a cryopreservative and cryoprotectant solution is used. The CAWS is kept frozen at −80° C. awaiting results of serial cultures until irradiation. C) Irradiation: Sterilization of the tissue lot with irradiation dose between 25 to 28 kGy, in dry ice for cold chain maintenance.

In a specific embodiment, the 10% glycerol is used as cryopreservative and cryoprotectant.

Then in step h) the validation, release, records are carried out: review of the donation, procurement and processing of the tissue, records in public tissue and organ transplant databases.

Finally, in the final stage, in step i) of transport, the processed tissue is transported to the generating center in optimal conditions for cold chain maintenance (−80° C.), and in j) the clinical use is informed to the tissue receptor about risks and benefits by obtaining his/her consent.

The present invention also includes the use of the CAWS of the present invention in the preparation or elaboration of a biological dressing useful in the treatment of chronic and complicated wounds to improve the wound bed for a posterior autograft, coverage defects with no conditions for autografts or local flaps, contained laparostomies without possibility of closure, and subsequent resection of melanoma.

### Examples

14 patients who requested, in a private consultation, an abdominoplasty to improve the esthetic appearance of the abdomen were evaluated, which when the evaluation process was completed, pre-surgical tests and informed consent for said surgery, they were invited to participate in donating skin from the resected abdominal fat dermis flap. The absence of tissue donation exclusion criteria was verified, they were subjected to a health survey, and routine tissue donation laboratory tests were taken (during surgery, so as not to have a window period), obtaining the signature of the informed consent of the tissue donation, being entered to the Integrated Organ Donation and Transplant System (SIDOT, by its acronym in spanish) in Chile. After donor selection, the process was divided into 10 steps: a) surgical/abdominoplasty process, b) procurement, c) packaging and identification, d) storage, e) transport to the processing center, f) processing, g) cryopreservation/quarantine, h) validation, release, records, i) transport, j) clinical use. All the processes followed the technical norm for the procurement, preservation and implantation of tissues of the Ministry of Health of Chile.
a. Surgical/Abdominoplasty Process: The procurement of skin was performed in a ward, at the same surgical time as the abdominoplasty, with all the asepsy and antisepsia measures, under general anesthesia and by the same surgical team (anesthetist, two plastic surgeons, procurement surgeon, surgeon's assistant, ward assistant, anesthesia assistant, ward nurse and procurement nurse). After marking the infraumbilical skin transverse ellipse (FIG. 1) and once the dissection of the abdominal adipose skin flap is performed, the redundant flap resection was performed, and then simultaneously dividing the teams and on the one hand to continue with the abdominoplasty and in an independent surgical table to perform the procurement of the skin.
b. Procuremente of the Skin: The resected adipose skin flap in the shape of an ellipse was placed on a separate surgical table. The procurement of the whole skin (including dermis) was performed, releasing the fat from the deeper dermis using scissors. Tissue samples (3) were also taken for current (aerobic) culture, anaerobic culture and mycotic culture The treated skin was deposited in a sterile container with 500 cc of physiological serum with 1 gr. of cloxacillin and 80 mg of gentamicin, closed in a hermetically sealed manner, taking care that the skin was completely submerged.
c. Packaging and Identification of the Skin: The skin containers were stored in a sterile double bag of at least 90 microns. Each one of them was hermetically closed, removing the most air and labeled with the tissue code and the date and time of the procurement.
d. Storage of the Skin: The skin was transported from the ward to the temporary storage location in a cooler with refrigerant units in order to maintain the temperature between 2 and 8° C.
e. Transportation of the Skin: The skin was sent by air to be processed from Iquique to the National Tissue Bank (BNT by its acronym in spanish) in Santiago, always before 36 hours of procurement, ensuring that the container was kept a temperature between 2 and 8° C.
f. Processing: The CAs were prepared in the BNT, as implants in two stages: a): flap measurement, segments cuts according to the requisition, review of the shave, wash cycles to decrease microbial load, sample collection number 2 for cultures (the first was intra-surgical) and immersion in cryopreservation solution for 1 hour. b) Preparation, trimming, measurement, packaging and labeling of each obtained film. Sample collection 3 and 4 (culture) during the packing stage. Sample collection 5 (culture) for backing.
g. Cryopreservation and Quarantine: 10% Glycerol is used as cryopreservative and cryoprotectant solution. The CAWS is kept frozen at −80° C. awaiting results of serial cultures until irradiation. c) Irradiation: Sterilization of the tissue lot with irradiation dose between 25 to 28 kGy, in dry ice for cold chain maintenance.
h. Validation, Release, Records: Review of the donation process, procurement and processing of tissue, records in SIDOT.
i. Transport: Return of processed tissue to the generating center in dry ice for cold chain maintenance (−80° C.).
j. Clinical use: The risks and benefits of the procedure were explained to the tissue receptor, obtaining the informed consent for the use of tissue from human origin, ensuring traceability and bio-surveillance through the tissue implant form with the receptor data and the amount of tissue used with the codes that identify it and possible adverse effects. The clinical indications for its use were: A) preparation of chronic and complicated wounds to improve the wound bed for a later autograft, b) defects of coverage with no conditions for autografts or local flaps, c) contained laparostomy without possibility of closure, and d) post-resection of melanoma. In the immediate pre-surgical period and once the size of the defect to be covered is calculated, the CAWS was washed 3 times with warm physiological serum (no more than 40° C.) to remove cryoprotectants. The receptor bed was prepared by scarectomy of necrotic, de-vitalized tissues and disordered granulation zones, fixing the CAWS with points and/or brackets associated with negative pressure therapy.

### Results

The sample of skin donors was composed of 14 female patients, aged between 31 and 55 years and an average age of 40 years, of whom 2 had a history of prior bariatric surgery. The procured cutaneous surface was estimated using the ellipse area formula, which subsequently decreased due to the primary contraction of the procured whole skin and the removal of edges at the time of processing to leave films of multiple sizes of clinical utility. The average number of processed skin and clinical utility films was 302 cm² and 8.3 films per patient, respectively.

The clinical sample was composed of 10 patients (2 procedures were performed in two patients), aged 2 months to 75 years, with diagnoses of: diabetic foot (4), contained laparostomy (2) lower extremity complex wound (2), recurrent sarcoma of the scalp (1), and melanoma (1). In all the patients there was an initial engraftment of CA, which after 21 days began the rejection evidenced by a change in the color of the CA and the formation of a superficial necrotic escar, which when it was removed had a vital tissue attached to the receptor, some patients were self-grafted, and others were managed with healing by second intention as definitive treatment. The histology of CA showed necrosis sites when it was removed with an infiltration mainly of nuclear polymorphs and for the case of the receptor bed an interface rich in fibroblasts and neoformation vessels. This interface or neo-dermis was visible by imaging, in the NMR of a patient with a diagnosis of diabetic foot and a history of transmetarsal amputation, the CA can be seen with a non-captant superficial component and a deep component that enhances with the contrast medium, simil to vascularized dermis. The results of the clinical series are summarized in the following table:

| Patient | Age | | Sex | Diagnostic | Evolution |
|---|---|---|---|---|---|
| 1 | 68 | years | Male | Diabetic foot. | Surgical cleaning/Bone |
| | | | | Transmetatarsal | resection/CAWS/Escarectomy/Autograft/ |
| | | | | amputation | Graft Loss/Advanced Cure/Total Closure. |
| 2 | 26 | years | Male | Complex wound leg | Surgical cleaning/CAWS/Advanced |
| | | | | | Healing. Healing and complete closure |
| 3* | 53 | years | Female | Recurrent | Resection/fenestration of skull/CAWS/ |
| | | | | dermatofibrosarcoma of | advanced healing. |
| | | | | the scalp | |
| 4 | 55 | years | Male | Diabetic foot. | Surgical cleaning/ |
| | | | | Transmetatarsal | CAWS/Escarectomy/Autograft/Loss of |
| | | | | amputation | follow-up |
| 5 | 70 | years | Male | Abdominal sepsis. | Complicated inguinal hernia, abdominal |
| | | | | Contained Laparostomy | sepsis, multiorgan failure, contained |
| | | | | | laparostomy, CAWS, clinical improvement, |
| | | | | | extubation. Death due to pneumonia |
| 6 | 2 | months | Male | Premature. Necrotizing | 30 weeks premature. Necrotizing |
| | | | | enterocolitis. Contained | enterocolitis, defunctionalization of |
| | | | | Laparostomy. | intestinal transit, evisceration (2 |
| | | | | | occasions). Contained |
| | | | | | Laparostomy/CAWS. Ostomal |
| | | | | | hernia/inguinal hernia. Periostomal and |
| | | | | | inguinal hernioplasty. (FIG. 8) |
| 7 | 60 | years | Male | Diabetic foot | Surgical cleaning/escarectomy/CAWS/ |
| | | | | | autograft/90% engraftment. Loss of follow- |
| | | | | | up |
| 8 | 49 | years | Male | Diabetic foot | Surgical cleaning/escarectomy/ |
| | | | | | CAWS/loss of follow-up |
| 9 | 57 | years | Male | Desforaging leg | Scarectomy/CAWS/autograft at 21 days. |
| | | | | | 100% Start Up. |
| 10 | 3 | months | Male | Premature. Necrotizing | 30 weeks premature. CAWS Rechange. |
| | | | | enterocolitis. Contained | Continent abdominal wall. Healing by |
| | | | | Laparostomy. | second intention. |
| 11 | 53 | years | Female | Recurrent | CAWS Rechange in central area. Without |
| | | | | dermatofibrosarcoma of | autologous graft due to the absence of |
| | | | | the scalp | central neodermis (initial bone wax in that |
| | | | | | area). |
| 12 | 75 | years | Male | Melanoma cheek | Melanoma resection. Awaiting for biopsy |
| | | | | | for possible margin |
| | | | | | expansion/reconstruction. |

For CAWS, the rejection is the natural evolution and in healthy individuals this occurs between 8 to 10 days, being delayed in large burns, due to depression of the immune system, between 15 to 30 days. In our experience with CAWSC, the rejection is later, being clinically evident from the third week, characterized by infiltration of nuclear polymorphs into the epidermis. The rejection of the CAWSC should not be interpreted as therapeutic failure, but rather a stage of what we have called intermediate coverage, since part of the CAWSC is lost and part of it adheres to the receptor bed, obtaining an interface of “neodermis”, which allows on the one hand, the final coverage to be completed with an autograft or on the other hand the healing to be completed by second intention, in which there is also a secondary contraction of the wound that decreases the size of the defect. In this way CAWSC can be used as an alternative to the use of dermal matrix, where in addition to the coverage of structures such as bone, cartilage and tendons, a better quality coating is obtained, with a higher thickness and elasticity 35.

The therapeutic uses of the CAWSC may be multiple, with the diabetic foot being highlighted in our series, deficit of extensive and complex coverages, contained laparostomies and subsequent resection of skin tumors, awaiting biopsies for eventual margin expansion and reconstruction.

### INDUSTRIAL APPLICATION

The present invention has application in the clinical industry, in the coverage of complex wounds, in most cases temporary but in some cases definitive, being an alternative to the use of dermal matrix.

1. Method for the elaboration of a live patch which allows the supply of tissues for skin transplants, of whole skin which includes the dermis, wherein the method comprises 3 main stages, namely:
i. obtaining whole skin tissue from a living donor,
ii. treatment and preservation of the tissue, obtaining a live patch, and finally
iii. tissue graft in the final patient.

2. Method according to claim 1, wherein stage i) of the method comprises steps a) surgical intervention, b) tissue procurement, c) packaging and identification, d) storage, and e) transport to a processing center.

3. Method according to claim 1, wherein stage ii) tissue treatment and preservation of tissue comprises steps f) processing, g) cryopreservation and quarantine, h) validation, records.

4. Method according to claim 1, wherein step iii) comprises steps i) transport and j) clinical use.

5. Method according to claim 2, wherein step a) is performed in ward, from where the tissue of the donor will be removed, where in a specific step a marking is made of the area where the tissue will be dissected, and once marked, dissection of the adipose skin tissue is performed, obtaining said adipose skin tissue, and the proper procurement is carried out.

6. Method according to claim 2, wherein step b) the tissue obtained in step a) is transported to a suitable surface where the procurement of the whole skin is performed, including the dermis, and releasing the fat from the deeper dermis using appropriate tools.

7. Method according to claim 6, wherein as quality control, in this step also tissue samples are taken to perform cultures of aerobic and anaerobic microorganisms, as well as mycotic cultures.

8. Method according to claim 2, wherein the tissue is deposited in a sterile container with physiological serum which is hermetically sealed, taking care that the tissue is completely immersed in serum.

9. Method according to claim 8, wherein the physiological serum contains antibiotics.

10. Method according to claim 2, wherein in stage c) the containers obtained in stage b) are stored in a sterile manner, labeling the container with data of at least one code assigned to the tissue, date and time of procurement.

11. Method according to claim 2, wherein in stage d) the tissue is stored in an appropriate container in order to maintain a temperature between 2 and 8° C., and subsequently, in stage e) the tissue is transported in a period less than 36 hours, and keeping the temperature in the range of 2 to 8° C.

12. Method according to claim 3, wherein once the tissue arrives a processing center, it undergoes the stages of:
1) Measurement of the live patch, segments cuts according to the requisition, review of the shave, wash cycles to decrease microbial load, sample collection number 2 for and immersion in cryopreservation solution for a period of 30 minutes to 3 hours,
2) Preparation, trimming, measurement, packaging and labeling of each obtained film, together with sample collection 3 and 4 (culture) during packing stage and sample collection 5 (culture) for backing, where in step g), cryopreservative and cryoprotectant solution is used for cryopreservation and quarantine, keeping the CAWS frozen at −80° C. awaiting results of serial cultures until irradiation.
3) Irradiation: Sterilization of the tissue lot with irradiation dose between 25 to 28 kGy, in dry ice for cold chain maintenance.

13. Method according to claim 12, wherein the cryopreservative and/or cryoprotectant is 10% glycerol.

14. Method according to claim 3, wherein in step h) validation, release, records are performed. review of the tissue donation process, procurement and processing of the tissue, records in public tissue and organ transplant databases.

15. Method according to claim 4, wherein in the final stage, in step i) of transport, the processed tissue is transported to the generating center in optimal conditions for cold chain maintenance (−80° C.), and in j) clinical use is informed to the tissue receptor about risks and benefits by obtaining his/her consent.

16. Use of a live patch elaborated according to the method of claims 1 to 15, wherein it serves for the preparation or elaboration of a useful patch in the treatment of chronic and complicated wounds to improve the wound bed for a subsequent autograft, coverage defects with no conditions for autografts or local flaps, contained laparostomies without possibility of closure, and subsequent resection of melanoma.
